(19) United States
(12) Patent Application Publication
(10) Pub. No.: US 2024/0241031 A1
(43) Pub. Date: Jul. 18, 2024

(54) Classification Model Generation Method, Particle Determination Method, and Recording Medium

(71) Applicants: ThinkCyte K.K., Tokyo (JP); The University of Tokyo, Tokyo (JP)

(72) Inventors: Hiroaki Adachi, Tokyo (JP); Issei Sato, Tokyo (JP)

(21) Appl. No.: 18/688,948

(22) PCT Filed: Aug. 29, 2022
(86) PCT No.: PCT/JP2022/032307

§ 371 Date: Mar. 4, 2024

(30) Foreign Application Priority Data
Sep. 17, 2021 (JP) 2021-152462

(51) Int. Cl. G01N 15/1429; G01N 15/01; G01N 15/10; G01N 15/1434; G01N 15/149

(52) U.S. Cl. CPC G01N 15/1429; G01N 15/01; G01N 15/1434; G01N 15/149; G01N 2015/1006

(57) ABSTRACT

In the classification model generation method, for each particle contained in a first sample containing a mixture of particles having specific morphological characteristics and other particles and a second sample that does not contain particles having the specific morphological characteristics but contains only the other particles, observation data indicating a result of observing the particle is acquired. By training using training data including the observation data and information indicating whether the observation data has been obtained from a particle contained in the first sample or the second sample, a classification model is generated that outputs discrimination information indicating whether or not a particle has the specific morphological characteristics when the observation data indicating a result of observing the particle is input.

### REFERENCE TO RELATED APPLICATIONS

This application is the national phase under 35 U. S. C. § 371 of PCT International Application No. PCT/JP2022/032307 which has an International filing date of Aug. 29, 2022 and designated the United States of America.

### BACKGROUND ART

Conventionally, a flow cytometry method has been used as a method for examining individual cells. The flow cytometry method is a cell analysis method for acquiring information regarding cells irradiated with light as a captured image or the like by making cells dispersed in a fluid pass through a channel, irradiating light to each cell moving through the channel, and measuring light such as scattered light or fluorescence from the cells irradiated with light. By using the flow cytometry method, a large number of cells can be examined one by one at high speed.

In addition, a ghost cytometry method (hereinafter, referred to as GC method) has been developed in which cells moving through a channel in the flow cytometer are irradiated with special structured illumination light, waveform data including compressed morphological information of cells is acquired from the cells, and the cells are classified based on the waveform data. An example of the GC method is disclosed in International Publication No. 2017/073737. In the GC method, a classification model is created in advance by machine learning from waveform data of cells prepared as training samples, and it is determined from the waveform data of cells contained in the training samples whether or not the cells are target cells to be classified by using the classification model. The flow cytometer using the GC method enables faster and more accurate cell analysis.

### SUMMARY

The flow cytometer using the GC method can discriminate cells having specific morphological characteristics without labeling, such as fluorescent staining. For example, when a disease causes a change in the morphology of a specific cell, the flow cytometer using the GC method can discriminate the cell that has changed its morphology among specimens collected from a person. Therefore, it is possible to find a patient with the disease. In order to create a classification model used in the GC method, it is necessary to prepare a target cell as a training sample in advance and acquire waveform data for the target cell. However, in some cases, such as when the target cell is a rare cell, it is not easy to acquire a large amount of waveform data for the target cell. For this reason, it may be difficult to discriminate cells having specific characteristics.

The present disclosure has been made in view of the above circumstances, and it is an object to provide a classification model generation method, a particle determination method, a computer program, and an information processing device for easily discriminating particles having specific morphological characteristics.

A classification model generation method according to an aspect of the present disclosure, is characterized by comprising: acquiring, for each particle contained in a first sample containing a mixture of particles having specific morphological characteristics and other particles and a second sample that does not contain particles having the specific morphological characteristics but contains only the other particles, observation data indicating a result of observing the particle: and generating a classification model, which outputs discrimination information indicating whether or not a particle has the specific morphological characteristics when the observation data indicating a result of observing the particle is input, by training using training data including the observation data and information indicating whether the observation data has been obtained from a particle contained in the first sample or the second sample.

In the classification model generation method according to an aspect of the present disclosure, it is characterized in that the observation data is waveform data indicating a temporal change in an intensity of light emitted from a particle irradiated with light by a structured illumination or waveform data indicating a temporal change in an intensity of light detected by structuring light from a particle irradiated with light.

In the classification model generation method according to an aspect of the present disclosure, it is characterized in that the first sample is a specimen collected from a person who has a specific disease, and the second sample is a specimen collected from a person who does not have the specific disease.

A particle determination method according to an aspect of the present disclosure, is characterized by comprising: acquiring observation data indicating a result of observing a particle: inputting the acquired observation data to a classification model, which outputs discrimination information indicating whether or not a particle has specific morphological characteristics when observation data indicating a result of observing the particle is input, and acquiring the discrimination information output from the classification model; and determining whether or not the particle related to the observation data has the specific morphological characteristics based on the acquired discrimination information, wherein the classification model is trained by using training data, which includes observation data indicating a result of observing a particle and information indicating whether the observation data has been obtained from a particle contained in a first sample or a second sample, for each particle contained in the first sample containing a mixture of particles having the specific morphological characteristics and other particles and the second sample that does not contain particles having the specific morphological characteristics but contains only the other particles.

The particle determination method according to an aspect of the present disclosure, is characterized by further comprising: outputting information regarding the particle for which the determination has been made.

In the particle determination method according to an aspect of the present disclosure, it is characterized in that a particle from which the observation data is to be acquired is collected from a person, a tag having identification information for identifying a person from whom a particle has been collected is attached to the particle, and when the particle related to the observation data has the specific morphological characteristics, a person from whom the particle has been collected is identified based on the identification information of the tag attached to the particle related to the observation data.

A computer program according to an aspect of the present disclosure, is characterized by causing a computer to execute processing of: acquiring, for each particle contained in a first sample containing a mixture of particles having specific morphological characteristics and other particles and a second sample that does not contain particles having the specific morphological characteristics but contains only the other particles, observation data indicating a result of observing the particle; and generating a classification model, which outputs discrimination information indicating whether or not a particle has the specific morphological characteristics when the observation data indicating a result of observing the particle is input, by training using training data including the observation data and information indicating whether the observation data has been obtained from a particle contained in the first sample or the second sample.

A computer program according to an aspect of the present disclosure, is characterized by causing a computer to execute processing of: acquiring observation data indicating a result of observing a particle: inputting the acquired observation data to a classification model, which outputs discrimination information indicating whether or not a particle has specific morphological characteristics when the observation data indicating a result of observing the particle is input, and acquiring the discrimination information output from the classification model: and determining whether or not the particle related to the observation data has the specific morphological characteristics based on the acquired discrimination information, wherein the classification model is trained by using training data, which includes observation data indicating a result of observing a particle and information indicating whether the observation data has been obtained from a particle contained in a first sample or a second sample, for each particle contained in the first sample containing a mixture of particles having the specific morphological characteristics and other particles and the second sample that does not contain particles having the specific morphological characteristics but contains only the other particles.

An information processing device according to an aspect of the present disclosure, is characterized by comprising: a data acquisition unit that acquires, for each particle contained in a first sample containing a mixture of particles having specific morphological characteristics and other particles and a second sample that does not contain particles having the specific morphological characteristics but contains only the other particles, observation data indicating a result of observing the particle: and a classification model generation unit that generates a classification model, which outputs discrimination information indicating whether or not a particle has the specific morphological characteristics when the observation data indicating a result of observing the particle is input, by training using training data including the observation data and information indicating whether the observation data has been obtained from a particle contained in the first sample or the second sample.

An information processing device according to an aspect of the present disclosure, is characterized by comprising: an observation unit that acquires observation data indicating a result of observing a particle: an discrimination information acquisition unit that inputs the acquired observation data to a classification model, which outputs discrimination information indicating whether or not a particle has specific morphological characteristics when the observation data indicating a result of observing the particle is input, and acquires the discrimination information output from the classification model; and a determination unit that determines whether or not the particle related to the observation data has the specific morphological characteristics based on the acquired discrimination information, wherein the classification model is trained by using training data, which includes observation data indicating a result of observing a particle and information indicating whether the observation data has been obtained from a particle contained in a first sample or a second sample, for each particle contained in the first sample containing a mixture of particles having the specific morphological characteristics and other particles and the second sample that does not contain particles having the specific morphological characteristics but contains only the other particles.

In one aspect of the present disclosure, a classification model is trained by using training data including observation data acquired for each particle contained in the first sample and the second sample. The first sample contains particles having specific morphological characteristics and other negative particles. The second sample does not contain particles having the specific morphological characteristics but contains only the other particles. The classification model outputs discrimination information indicating whether or not the particle has the specific morphological characteristics when observation data is input. Even if particles having the specific morphological characteristics are rare and accordingly it is not easy to acquire a large amount of observation data, it is possible to generate a classification model by training using training data including the observation data and information indicating whether the observation data has been obtained from a particle contained in the first sample or the second sample. Using the classification model, it is possible to obtain discrimination information according to the observation data and determine whether or not the particle has specific morphological characteristics based on the discrimination information.

In one aspect of the present disclosure, the observation data is waveform data indicating a temporal change in the intensity of light emitted from a particle irradiated with light by the structured illumination or waveform data indicating a temporal change in the intensity of light detected by structuring light from a particle irradiated with light. The waveform data is similar to that used in the GC method, and includes compressed morphological information of particles. Therefore, the waveform data can be used for generating a classification model and determining particles.

In one aspect of the present disclosure, the first sample is a specimen collected from a person who has a specific disease such as cancer, and the second sample is a specimen collected from a person who does not have the specific disease. A particle having specific morphological characteristics is a particle (positive particle) whose morphology has changed due to a specific disease such as cancer. By determining whether or not the particle contained in the sample is a positive particle, it is possible to determine whether or not the person from whom the particle has been collected has a specific disease.

In one aspect of the present disclosure, information regarding the determined particle is displayed. The user can check the information regarding the particle.

In one aspect of the present disclosure, a tag having identification information for identifying a person from whom a particle has been collected is attached to the particle, and the person from whom the particle has been collected is identified based on the tag. Based on the tag attached to the particle having the specific morphological characteristics, it is possible to identify a person from whom the particle having the specific morphological characteristics has been collected. Therefore, for example, it is also possible to identify a person having a specific disease among persons from whom particles have been collected.

In one aspect of the present disclosure, even if it is not easy to acquire observation data by preparing a large number of training samples containing particles with specific morphological characteristics, it is possible to generate a classification model that outputs discrimination information indicating whether or not a particle is the particle having specific morphological characteristics in response to the input of the observation data. In the present disclosure, excellent effects are obtained, such as being able to discriminate particles having specific morphological characteristics by using a classification model.

The above and further objects and features will more fully be apparent from the following detailed description with accompanying drawings.

### BRIEF DESCRIPTION OF DRAWINGS

FIG. 1 is a conceptual diagram showing an overview of a classification model generation method.

FIG. 2 is a block diagram showing a configuration example of a training apparatus according to a first embodiment for generating a classification model.

FIG. 3 is a graph showing an example of waveform data.

FIG. 4 is a block diagram showing an example of the internal configuration of an information processing device.

FIG. 5 is a conceptual diagram showing the functions of a classification model.

FIG. 6 is a flowchart showing an example of a procedure of processing for training a classification model.

FIG. 7 is a schematic diagram showing an example of training using the concept of MIL.

FIG. 8 is a conceptual diagram showing an overview of a particle determination method.

FIG. 9 is a block diagram showing a configuration example of a determination apparatus according to the first embodiment for determining cells.

FIG. 10 is a block diagram showing an example of the internal configuration of an information processing device.

FIG. 11 is a flowchart showing the procedure of processing performed by an information processing device to determine cells.

FIG. 12 is a schematic diagram showing a display example of determination results.

FIG. 13 is a block diagram showing a configuration example of a training apparatus according to a second embodiment.

FIG. 14 is a block diagram showing a configuration example of a determination apparatus according to the second embodiment.

FIG. 15 is a block diagram showing a configuration example of a training apparatus according to a third embodiment.

FIG. 16 is a block diagram showing a configuration example of a determination apparatus according to the third embodiment.

### DESCRIPTION

Hereinafter, the present disclosure will be specifically described with reference to the diagrams showing embodiments thereof.

### First Embodiment

In the present embodiment, using the GC method, it is determined whether or not a cell has specific morphological characteristics based on waveform data indicating the morphological characteristics of the cell obtained by irradiating light to the cell. The waveform data corresponds to observation data. Hereinafter, the present embodiment will be described by using an example of discriminating cells having specific morphological characteristics due to the influence of a specific disease, such as cancer cells, among a plurality of cells contained in specimens collected from a person.

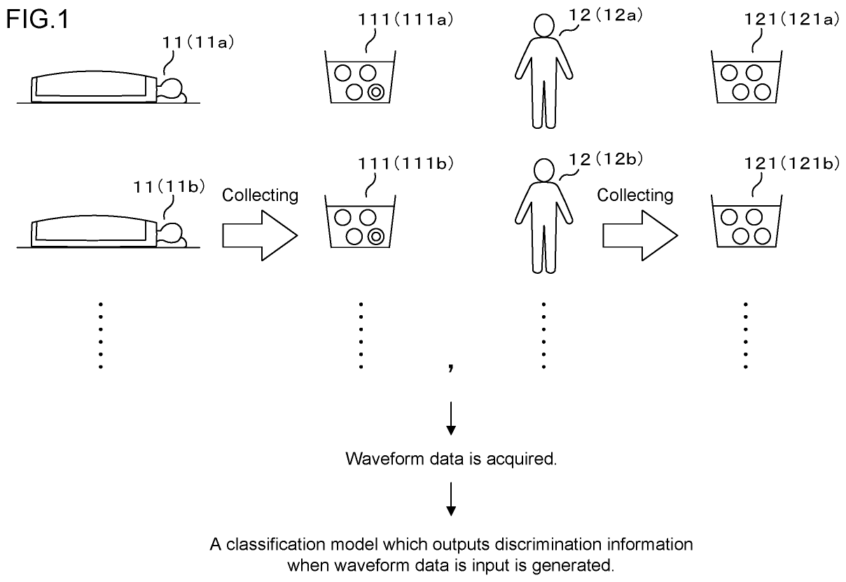

In the present embodiment, a classification model necessary for discriminating cells is generated. The classification model is a trained model. FIG. 1 is a conceptual diagram showing an overview of a classification model generation method. A plurality of first samples 111 are created in which cells having specific morphological characteristics and other cells are mixed, and a plurality of second samples 121 are created that do not contain cells having the specific morphological characteristics and contain only the other cells. For example, the first sample 111 is a specimen collected from a patient 11 who has a specific disease such as cancer, and the second sample 121 is a specimen collected from a healthy person 12 who does not have the specific disease. The symbols in parentheses shown in FIG. 1 refer to each patient 11 and each first sample 111, and also refer to each healthy person 12 and each second sample 121. That is, in the example shown in FIG. 1, a first sample 111a is collected from a patient 11a, and a first sample 111b is collected from a patient 11b. In addition, a second sample 121a is collected from a healthy person 12a, and a second sample 121b is collected from a healthy person 12b. The first sample 111 contains cells having specific morphological characteristics due to the influence of a specific disease, such as cancer cells, and other cells. The other cells are cells that do not have the specific morphological characteristics. The second sample 121 contains only other cells. Hereinafter, cells having specific morphological characteristics will be referred to as positive cells, and other cells will be referred to as negative cells. In FIG. 1, a positive cell is indicated by double circles, and a negative cell is indicated by a circle. There are many types of negative cells. Usually, positive cells are cells that are rarely present. The number of positive cells in the first sample 111 is usually smaller than the number of negative cells.

Then, each cell contained in the first sample 111 and the second sample 121 is irradiated with light, and waveform data indicating the morphological characteristics of the cell is acquired from each cell. As will be described later, the waveform data indicates a temporal change in the intensity of light emitted from the cell irradiated with light, and the waveform data includes the morphological characteristics of the cell. Then, a classification model is generated by training using training data that includes waveform data and information indicating whether the waveform data has been obtained from cells contained in the first sample or the second sample. The classification model outputs discrimination information indicating whether or not a cell has specific morphological characteristics when waveform data is input.

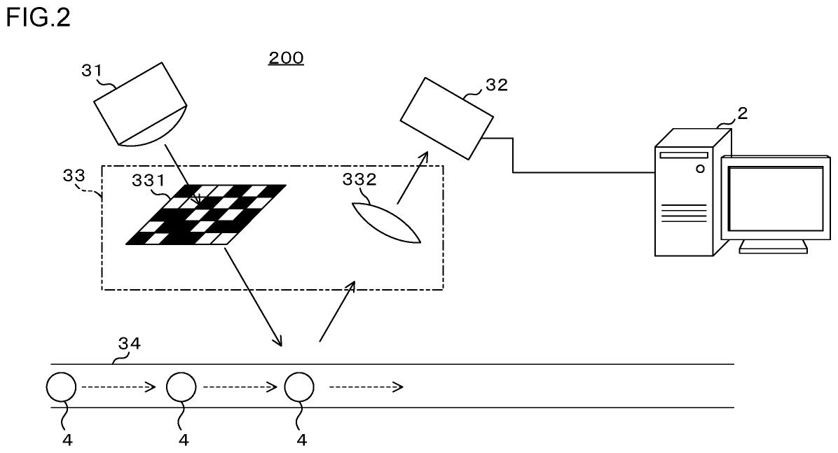

FIG. 2 is a block diagram showing a configuration example of a training apparatus 200 according to the first embodiment for generating a classification model. The training apparatus 200 includes a channel 34 through which cells flow. Cells 4 are dispersed in the fluid, and as the fluid flows through the channel 34, the individual cells 4 sequentially move through the channel 34. The training apparatus 200 includes a light source 31 that irradiates light to the cells 4 moving through the channel 34. The light source 31 irradiates white light or monochromatic light. The light source 31 is, for example, a laser light source or an LED (Light Emitting Diode) light source. The cell 4 irradiated with light emits light. The light emitted from the cell 4 is, for example, reflected light, scattered light, transmitted light, fluorescence, Raman scattered light, or diffracted light thereof. The training apparatus 200 includes a detection unit 32 that detects light from the cell 4. The detection unit 32 includes a light detection sensor such as a photomultiplier tube (PMT), a line type PMT element, a photodiode, an APD (Avalanche Photo-Diode), or a semiconductor optical sensor. In FIG. 2, the path of light is indicated by solid arrows.

The training apparatus 200 includes an optical system 33. The optical system 33 guides illumination light from the light source 31 to the cell 4 in the channel 34, and causes the light from the cell 4 to be incident on the detection unit 32. The optical system 33 includes a spatial light modulation device 331 for modulating and structuring the incident light. The training apparatus 200 shown in FIG. 2 is configured such that the illumination light from the light source 31 is irradiated to the cell 4 through the spatial light modulation device 331. The spatial light modulation device 331 is a device for modulating light by controlling the spatial distribution (amplitude, phase, polarization, and the like) of light. The spatial light modulation device 331 has, for example, a plurality of regions on a surface on which light is incident, and the incident light is modulated differently in two or more of the plurality of regions. Here, modulation means changing the characteristics of light (any one or more of light properties such as intensity, wavelength, phase, and polarization state of light). In FIG. 2, a configuration is exemplified in which two types of regions having different light transmittances are arranged in a predetermined pattern of a two-dimensional grid pattern. In FIG. 2, the illumination light from the light source 31 passes through the spatial light modulation device 331 to form structured illumination in which two types of light having different intensities are arranged in a predetermined pattern.

The spatial light modulation device 331 is, for example, a diffractive optical element (DOE), a spatial light modulator (SLM), or a digital micromirror device (DMD). When the illumination light emitted from the light source 31 is incoherent light, the spatial light modulation device 331 is a DMD. Another example of the spatial light modulation device 331 is a film or optical filter in which a plurality of types of regions having different light transmittances are arranged randomly or in a predetermined pattern. Here, the plurality of types of regions having different light transmittances are arranged in a predetermined pattern means, for example, a state in which a plurality of types of regions having different light transmittances are arranged in a one-dimensional or two-dimensional grid pattern. In addition, a plurality of types of regions having different light transmittances are arranged randomly means that the plurality of types of regions are arranged so as to be irregularly scattered. The film or optical filter described above has at least two types of regions: regions having a first light transmittance and regions having a second transmittance different from the first light transmittance. Thus, the illumination light from the light source 31 is modulated by the spatial light modulation device 331 before being irradiated to the cell 4. For example, the illumination light from the light source 31 is converted into structured illumination light in which bright spots with different light intensities are arranged randomly or in a predetermined pattern. Thus, the configuration in which the illumination light from the light source 31 is modulated by the spatial light modulation device 331 in the middle of the optical path from the light source 31 to irradiation to the cell 4 is also referred to as a structured illumination.

The illumination light by the structured illumination is irradiated to a specific region (irradiation region) in the channel 34. When the cell 4 moves within the irradiation region, the cell 4 is irradiated with the structured illumination light. Although the pattern of the structured illumination light emitted to the cell 4 is constant and does not change over time, the cell 4 is irradiated with light having different light intensities depending on the location as the cell 4 moves through the irradiation region. The cell 4 is irradiated with the structured illumination light and emits light, such as transmitted light, fluorescence, scattered light, interference light, diffracted light, or polarized light, which is emitted from the cell 4 or generated through the cell 4. Hereinafter, the light emitted from the cell 4 or generated through the cell 4 will also be referred to as light modulated by the cell 4. The light modulated by the cell 4 is detected by the detection unit 32 continuously while the cell 4 passes through the irradiation region of the channel 34. The detection unit 32 outputs a signal according to the intensity of the detected light to an information processing device 2. In this manner, the training apparatus 200 can acquire waveform data indicating a temporal change in the intensity of light modulated by the cell 4 that has been detected by the detection unit 32.

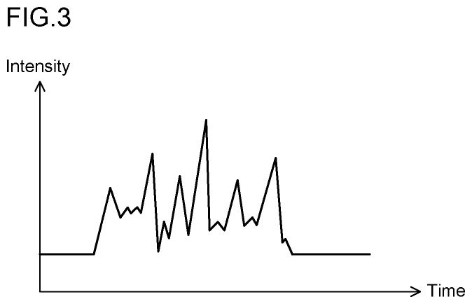

FIG. 3 is a graph showing an example of waveform data. In FIG. 3, the horizontal axis indicates time, and the vertical axis indicates the intensity of light detected by the detection unit 32. The waveform data includes a plurality of intensity values obtained sequentially over time. Each intensity value indicates the intensity of light. The waveform data herein is time-series data indicating a temporal change in the optical signal that reflects the morphological characteristics of the cell 4. The optical signal is a signal indicating the intensity of light detected by the detection unit 32. The waveform data is, for example, waveform data that is obtained by using the GC method and indicates a temporal change in the intensity of light emitted from the cell 4. The optical signal from the cell 4 obtained by using the GC method includes compressed morphological information of the cell. Therefore, the temporal change in the intensity of the light detected by the detection unit 32 varies according to the morphological characteristics of the cell 4, such as the size, shape, internal structure, density distribution, or color distribution. The intensity of the light from the cell 4 also varies with the temporal changes in the intensity of the structured illumination light over time as the cell 4 moves within the irradiation region in the channel 34. As a result, the intensity of the light detected by the detection unit 32 changes over time, and the intensity of the light that changes over time forms a waveform on the graph as shown in FIG. 3.

The waveform data indicating the temporal change in the intensity of light modulated by the cell 4, which is obtained by the structured illumination, is waveform data including compressed morphological information according to the morphological characteristics of the cell 4. For this reason, in the flow cytometer using the GC method, morphologically different cells are discriminated by machine learning using the waveform data as training data as it is. In addition, it is also possible to generate images of the cell 4 from the waveform data obtained by the structured illumination. In addition, the training apparatus 200 may be configured to individually acquire waveform data for a plurality of types of modulated light emitted from one cell 4.

The optical system 33 includes a lens 332 in addition to the spatial light modulation device 331. The lens 332 collects the light from the cell 4 and makes the collected light incident on the detection unit 32. In addition to the spatial light modulation device 331 and the lens 332, the optical system 33 includes optical components, such as a mirror, a lens, and a filter, in order to structure the illumination light from the light source 31, irradiate the cell 4 with the structured illumination light, and make the light from the cell 4 incident on the detection unit 32. In FIG. 2, descriptions of optical components that can be included in the optical system 33 other than the spatial light modulation device 331 and the lens 332 are omitted. The waveform data indicating a temporal change in the intensity of light from the cell 4 acquired by the detection unit 32 is observation data including morphological information of the cell.

The training apparatus 200 includes the information processing device 2. The information processing device 2 performs information processing necessary for generating a classification model. The detection unit 32 is connected to the information processing device 2. The detection unit 32 outputs a signal according to the intensity of the detected light to the information processing device 2, and the information processing device 2 receives the signal from the detection unit 32.

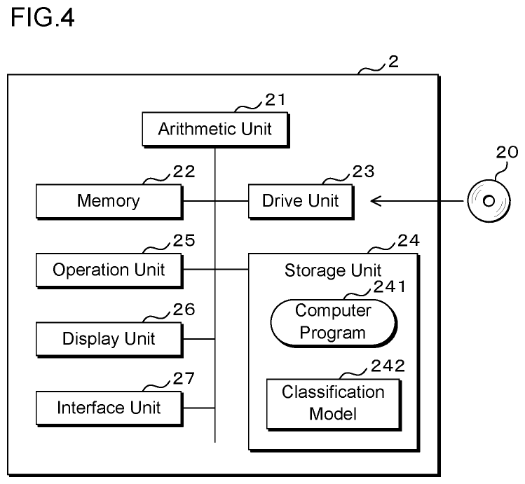

FIG. 4 is a block diagram showing an example of the internal configuration of the information processing device 2. The information processing device 2 is, for example, a computer such as a personal computer or a server device. The information processing device 2 includes an arithmetic unit 21, a memory 22, a drive unit 23, a storage unit 24, an operation unit 25, a display unit 26, and an interface unit 27. The arithmetic unit 21 is configured by using, for example, a CPU (Central Processing Unit), a GPU (Graphics Processing Unit), or a multi-core CPU. The arithmetic unit 21 may be configured by using a quantum computer. The memory 22 stores temporary data generated along with calculations. The memory 22 is, for example, a RAM (Random Access Memory). The drive unit 23 reads information from a recording medium 20 such as an optical disc or a portable memory.

The storage unit 24 is nonvolatile, and is, for example, a hard disk or a nonvolatile semiconductor memory. The operation unit 25 receives an input of information, such as text, by receiving an operation from the user. The operation unit 25 is, for example, a touch panel, a keyboard, or a pointing device. The display unit 26 displays an image. The display unit 26 is, for example, a liquid crystal display or an EL display (Electroluminescent Display). The operation unit 25 and the display unit 26 may be integrated. The interface unit 27 is connected to the detection unit 32. The interface unit 27 transmits and receives signals to and from the detection unit 32.

The arithmetic unit 21 causes the drive unit 23 to read a computer program 241 recorded on the recording medium 20, and to store the read computer program 241 in the storage unit 24. The arithmetic unit 21 performs processing necessary for the information processing device 2 according to the computer program 241. The computer program 241 may be downloaded from the outside of the information processing device 2. Alternatively, the computer program 241 may be stored in the storage unit 24 in advance. In these cases, the information processing device 2 does not need to include the drive unit 23. In addition, the information processing device 2 may be configured by a plurality of computers.

The information processing device 2 includes a classification model 242. The classification model 242 is realized by the arithmetic unit 21 executing information processing according to the computer program 241. The storage unit 24 stores data necessary for realizing the classification model 242. The classification model 242 may be configured by using hardware. The classification model 242 may be realized by using a quantum computer. Alternatively, the classification model 242 may be provided outside the information processing device 2, and the information processing device 2 may perform processing by using the external classification model 242. For example, the classification model 242 may be configured on the cloud.

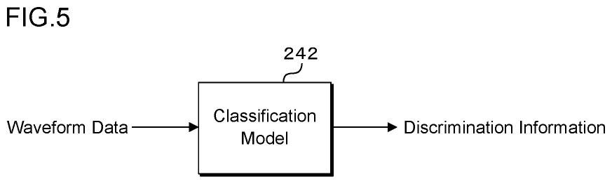

FIG. 5 is a conceptual diagram showing the functions of the classification model 242. Waveform data obtained from one cell 4 is input to the classification model 242. The classification model 242 is trained to output discrimination information indicating whether or not the cell 4 has specific morphological characteristics when the waveform data is input. For example, the classification model 242 is configured by a neural network.

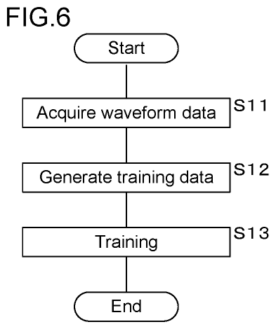

The information processing device 2 executes a classification model generation method by performing processing for training the classification model 242. FIG. 6 is a flowchart showing an example of the procedure of processing for training the classification model 242. Hereinafter, the step is abbreviated as S. The arithmetic unit 21 performs the following processing according to the computer program 241. Before the classification model 242 is trained, a plurality of first samples 111 and a plurality of second samples 121 are created. The information processing device 2 acquires waveform data obtained from each cell contained in the plurality of first samples 111 and waveform data obtained from each cell contained in the plurality of second samples 121 (S11).

When the processing of S11 is performed, each cell 4 contained in the plurality of first samples 111 flows through the channel 34, and structured illumination light is irradiated to the cell 4 by using the light source 31 and the spatial light modulation device 331. The cell 4 emits light modulated by the cell 4, such as scattered light, and the emitted light is detected by the detection unit 32. The detection unit 32 outputs a signal according to the intensity of the detected light to the information processing device 2, and the information processing device 2 receives the signal from the detection unit 32 by using the interface unit 27. The arithmetic unit 21 acquires waveform data by generating waveform data indicating a temporal change in the intensity of the light detected by the detection unit 32 based on the signal from the detection unit 32. In this manner, waveform data for each cell 4 contained in the plurality of first samples 111 is acquired by the information processing device 2. Similarly, waveform data for each cell 4 contained in the plurality of second samples 121 is acquired by the information processing device 2. The arithmetic unit 21 stores the acquired waveform data in the storage unit 24. The processing of S11 corresponds to a data acquisition unit.

Then, the information processing device 2 generates training data for training (S12). The training data includes waveform data and information indicating whether the waveform data has been obtained from the cell contained in the first sample 111 or the second sample 121. The waveform data obtained from the cell contained in the first sample 111 is associated with information indicating that the waveform data has been obtained from the cell contained in the first sample 111. The waveform data obtained from the cell contained in the second sample 121 is associated with information indicating that the waveform data has been obtained from the cell contained in the second sample 121. In S12, the arithmetic unit 21 generates training data by associating each piece of waveform data with information indicating whether the waveform data has been obtained from the cell contained in the first sample 111 or the second sample 121. Waveform data obtained from a plurality of cells contained in the first sample 111 may be similarly associated with information indicating that the waveform data has been obtained from the first sample 111 regardless of whether each cell is a positive cell or a negative cell, and this information may be included in the training data. Similarly, waveform data obtained from a plurality of cells contained in the second sample 121 may be associated with information indicating that the waveform data has been obtained from the second sample 121, and this information may be included in the training data. The arithmetic unit 21 stores training data in the storage unit 24.

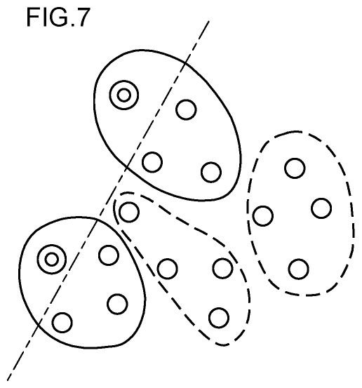

Then, the information processing device 2 trains the classification model 242 (S13). In S13, the arithmetic unit 21 performs training using a MIL (Multiple-Instance Learning) method. The MIL method is disclosed, for example, in Marc-Andre Carbonneau, et al. “Multiple instance learning: A survey of problem characteristics and applications”, Pattern Recognition, Volume 77, May 2018, Pages 329 to 353. FIG. 7 is a schematic diagram showing an example of training using the concept of MIL. It is assumed that the characteristics of cells are plotted on two-dimensional coordinates. In FIG. 7, a positive cell having specific morphological characteristics is indicated by double circles, and a negative cell is indicated by a circle. A plurality of cells surrounded by the solid line are a plurality of cells contained in the same first sample 111. On the other hand, a plurality of cells surrounded by the dashed line are a plurality of cells contained in the same second sample 121. The dashed-dotted line in FIG. 7 indicates a decision boundary when classifying positive cells and negative cells based on specific morphological characteristics. In the MIL, a classification model is trained to appropriately set the boundary. A more reasonable decision boundary is set by training so that all of a plurality of cells contained in each second sample 121 are negative cells and that some cells having specific morphological characteristics contained in each first sample 111 are positive cells and the other cells are negative cells.

In S13, the arithmetic unit 21 adjusts the calculation parameters of the classification model 242 so that discrimination information indicating that the cell is not a positive cell having specific morphological characteristics is output when the waveform data obtained from the cell contained in the second sample 121 is input to the classification model 242. In addition, the arithmetic unit 21 adjusts the calculation parameters of the classification model 242 so that discrimination information indicating that the cell is a positive cell is output when some of the waveform data obtained from a plurality of cells contained in one first sample 111 are input to the classification model 242 and discrimination information indicating that the cell is not a positive cell is output when the other pieces of the waveform data are input to the classification model 242.

The arithmetic unit 21 trains the classification model 242 by repeating the processing for adjusting the calculation parameters of the classification model 242 using the training data. When the classification model 242 is a neural network, the calculation parameters of each node are adjusted. The classification model 242 is trained to output discrimination information indicating that the cell is a positive cell when waveform data obtained from some cells contained in the first sample 111 is input and to output discrimination information indicating that the cell is not a positive cell when waveform data obtained from the other cells contained in the first sample 111 and cells contained in the second sample 121 are input. The arithmetic unit 21 stores learned data, in which the adjusted final parameters are recorded, in the storage unit 24. In this manner, the trained classification model 242 is generated. The processing of S13 corresponds to a classification model generation unit. After S13 ends, the information processing device 2 ends the process for training the classification model 242.

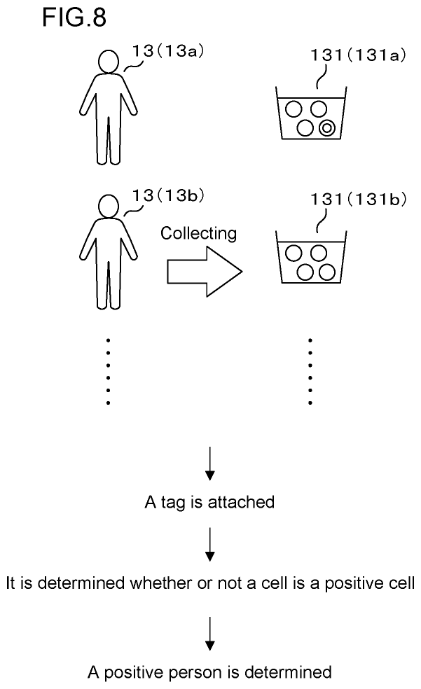

By generating the trained classification model 242 and determining whether the cell is a positive cell having specific morphological characteristics, a particle determination method is executed. In addition, in the particle determination method, a specimen collected from a person is used as a sample, it is determined whether or not each cell contained in the sample is a positive cell by using the classification model 242, and the person whose specimen has been found to have a cell determined to be a positive cell is identified as a positive person. FIG. 8 is a conceptual diagram showing an overview of the particle determination method. For example, a specimen is collected from a test subject 13 such as a person undergoing cancer screening, and the specimen is used as a test sample 131. The specimen is blood, a fractionated component containing specific cells generated from blood, urine, bone marrow fluid or other body fluids, a cleaning solution obtained after cleaning the affected area, and the like, and contains cells. The test sample 131 contains one or more cells. By collecting specimens from a plurality of test subjects 13, a plurality of test samples 131 are created. The symbols in parentheses shown in FIG. 8 refer to each test subject 13 and each test sample 131. That is, in the example shown in FIG. 8, a test sample 131a is collected from a test subject 13a, and a test sample 131b is collected from a test subject 13b.

Then, a tag having identification information for identifying each test subject 13 is attached to the cell contained in the test sample 131. For example, in the example shown in FIG. 8, a tag having identification information for identifying the test subject 13a is attached to the cell contained in the test sample 131a. The tag having identification information connects each cell contained in the test sample 131 and the test subject 13 from whom the cells have been collected, and is preferably attached or bonded to the cell itself. The tag is preferably a substance containing a constituent element formed by linking a plurality of types of components, such as a known peptide tag or a DNA (deoxyribonucleic acid) tag. The sequence of the constituent element contained in the tag indicates identification information. More preferably, the tag is a DNA tag. For example, in the DNA tag, the base sequence indicates identification information. The identification information of the tag attached to the cell differs depending on each test sample 131, and is associated with the test subject 13. For example, in the example shown in FIG. 8, the test sample 131a is associated with the test subject 13a. As a DNA tag for adding identification information to each cell, for example, oligonucleotides modified with lipids or cholesterol as described in WO2020/010366 can be used.

Then, using the classification model 242, it is determined whether or not each cell contained in the test sample 131 is a positive cell. When the cell is determined to be a positive cell by the classification model 242, the test subject 13 from whom the test sample 131 containing the positive cell has been collected is then identified based on the identification information of the tag attached to the positive cell, and the identified test subject 13 is determined to be a positive person. For example, in the example shown in FIG. 8, the test subject 13a from whom the test sample 131a containing the positive cell has been collected is determined to be a positive person.

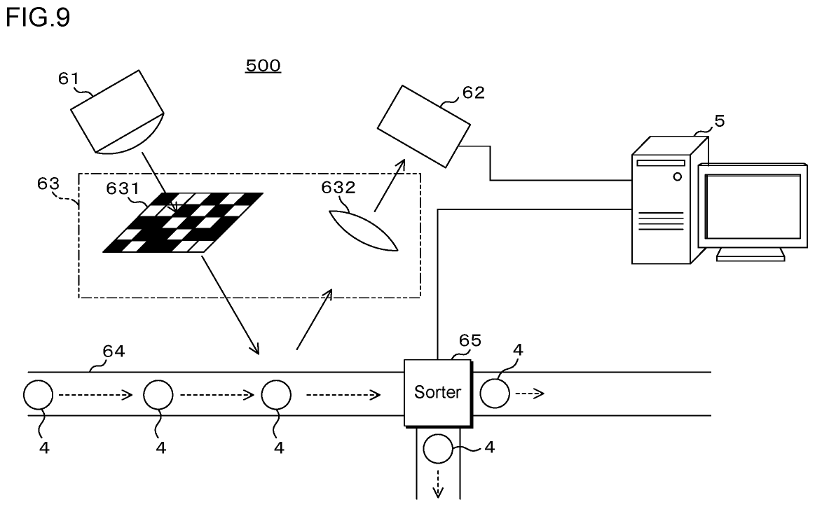

FIG. 9 is a block diagram showing a configuration example of a determination apparatus 500 according to the first embodiment for determining cells. The determination apparatus 500 includes a channel 64 for cells. The cells 4 sequentially move through channel 64. The determination apparatus 500 includes a light source 61, a detection unit 62, and an optical system 63. The configurations of the light source 61, the detection unit 62, and the optical system 63 in FIG. 9 are the same as the configurations of the light source 31, the detection unit 32, and the optical system 33 in FIG. 2. The light source 61 is, for example, a laser light source or an LED light source, and the detection unit 62 has a light detection sensor such as a photomultiplier tube, a line type PMT element, a photodiode, an APD, or a semiconductor optical sensor. In FIG. 9, the path of light is indicated by solid arrows.

The optical system 63 guides the light from the light source 61 to the cell 4 in the channel 64 and causes the light from the cell 4 to enter the detection unit 62. The optical system 63 includes a spatial light modulation device 631 and a lens 632. The light from the light source 61 passes through the spatial light modulation device 631 and is then irradiated to the cell 4. This forms the structured illumination. The determination apparatus 500 can acquire waveform data indicating a temporal change in the intensity of light emitted from the cell 4 and modulated by the cell 4. The waveform data is used, for example, in the GC method, and indicates the morphological characteristics of the cell 4. The determination apparatus 500 may be configured to acquire a plurality of types of waveform data for one cell 4.

It is preferable that the optical system 63 includes optical components for irradiating the cell 4 with light from the light source 61 and making the light from the cell 4 incident on the detection unit 62, such as a mirror, a lens, and a filter, in addition to the spatial light modulation device 631 and the lens 632. In FIG. 9, descriptions of optical components other than the spatial light modulation device 631 and the lens 632 are omitted. The waveform data indicating the temporal change in the intensity of light from the cell 4 acquired by the detection unit 62 is observation data including the morphological information of the cell.

A sorter 65 is connected to the channel 64. The sorter 65 sorts specific cells from the cell 4 that has moved through the channel 64. For example, when the cell 4 that has moved through the channel 64 is determined to be the specific cell, the sorter 65 applies an electric charge to the moving cell 4 and applies a voltage to change the movement path of the cell 4, thereby sorting the cell 4. The sorter 65 may be configured to generate a pulse flow when the cell 4 flows to the sorter 65 and change the movement path of the cell 4 to sort the specific cell 4.

The determination apparatus 500 includes an information processing device 5. The information processing device 5 performs information processing necessary for determining the cell 4. The detection unit 62 is connected to the information processing device 5. The detection unit 62 outputs a signal according to the intensity of the detected light to the information processing device 5, and the information processing device 5 receives the signal from the detection unit 62. The sorter 65 is connected to the information processing device 5 and is controlled by the information processing device 5. The sorter 65 sorts cells under the control of the information processing device 5.

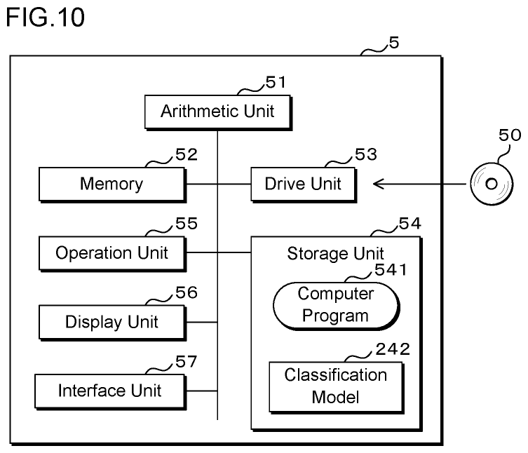

FIG. 10 is a block diagram showing an example of the internal configuration of the information processing device 5. The information processing device 5 is a computer such as a personal computer or a server device. The information processing device 5 includes an arithmetic unit 51, a memory 52, a drive unit 53, a storage unit 54, an operation unit 55, a display unit 56, and an interface unit 57. The arithmetic unit 51 is configured by using, for example, a CPU, a GPU, or a multi-core CPU. The arithmetic unit 51 may be configured by using a quantum computer. The memory 52 stores temporary data generated along with calculations. For example, the drive unit 53 reads information from a recording medium 50 such as an optical disc.

The storage unit 54 is nonvolatile, and is, for example, a hard disk or a nonvolatile semiconductor memory. The operation unit 55 receives an input of information, such as text, by receiving an operation from the user. The operation unit 55 is, for example, a touch panel, a keyboard, or a pointing device. The display unit 56 displays an image. The display unit 56 is, for example, a liquid crystal display or an EL display. The operation unit 55 and the display unit 56 may be integrated. The interface unit 57 is connected to the detection unit 62 and the sorter 65. The interface unit 57 transmits and receives signals to and from the detection unit 62 and the sorter 65.

The arithmetic unit 51 causes the drive unit 53 to read a computer program 541 recorded on the recording medium 50, and stores the read computer program 541 in the storage unit 54. The arithmetic unit 51 performs processing necessary for the information processing device 5 according to the computer program 541. In addition, the computer program 541 may be downloaded from the outside of the information processing device 5. Alternatively, the computer program 541 may be stored in the storage unit 54 in advance. In these cases, the information processing device 5 does not need to include the drive unit 53. In addition, the information processing device 5 may be configured by a plurality of computers.

The information processing device 5 includes the classification model 242. The classification model 242 is realized by the arithmetic unit 51 executing information processing according to the computer program 541. The classification model 242 is a classification model trained by the training apparatus 200. The information processing device 5 includes the classification model 242 by storing learned data, in which the parameters of the classification model 242 trained by the training apparatus 200 are recorded, in the storage unit 54. For example, the learned data is read from the recording medium 50 by the drive unit 53 or downloaded. The classification model 242 may be configured by hardware. The classification model 242 may be realized by using a quantum computer. Alternatively, the classification model 242 may be provided outside the information processing device 5, and the information processing device 5 may perform processing by using the external classification model 242. For example, the classification model 242 may be configured on the cloud.

In the information processing device 5, the classification model 242 may be realized by an FPGA (Field Programmable Gate Array). The circuit of the FPGA is configured based on the parameters of the classification model 242 trained by using the classification model generation method, and the FPGA executes the processing of the classification model 242.

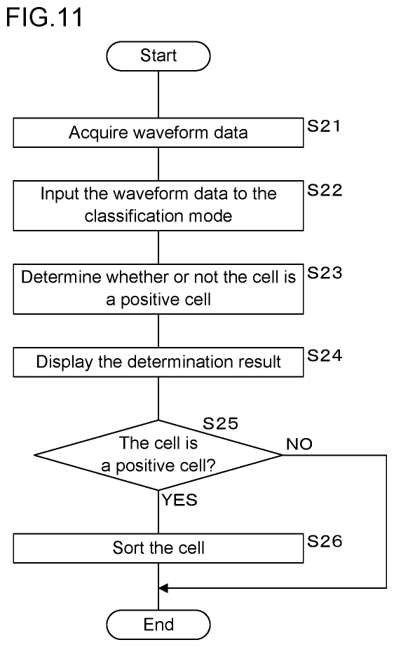

FIG. 11 is a flowchart showing the procedure of processing performed by the information processing device 5 to determine cells. The arithmetic unit 51 performs the following processing according to the computer program 541. The test sample 131 is created as described above, and a tag is attached to each cell contained in the test sample 131. The tagged cell 4 is allowed to flow through the channel 64. The information processing device 5 acquires waveform data obtained from the cell 4 moving through the channel 64 (S21). The cell 4 moving through the channel 64 is irradiated with light by the structured illumination, light emitted from the cell 4 is detected by the detection unit 62, the detection unit 62 outputs a signal according to the detection to the information processing device 5, and the information processing device 5 receives the signal. In S21, the arithmetic unit 51 generates waveform data indicating a temporal change in the intensity of light detected by the detection unit 62 based on the signal from the detection unit 62, and stores the waveform data in the storage unit 54. The processing of S21 corresponds to an observation unit.

The information processing device 5 inputs the acquired waveform data to the classification model 242 (S22). In S22, the arithmetic unit 51 inputs the waveform data to the classification model 242, and causes the classification model 242 to perform processing. The arithmetic unit 51 does not input information indicating whether the waveform data has been obtained from the cell contained in the first sample or the second sample. In response to the input of the waveform data, the classification model 242 performs processing for outputting discrimination information indicating whether or not the cell 4 is a positive cell having specific morphological characteristics. The arithmetic unit 51 acquires the discrimination information output from the classification model 242. The processing of S22 corresponds to a discrimination information acquisition unit. The information processing device 5 determines whether or not the cell 4 is a positive cell based on the discrimination information output from the classification model 242 (S23). In S23, the arithmetic unit 51 determines that the cell 4 is a positive cell when the discrimination information indicates that the cell 4 is a positive cell, and determines that the cell 4 is not a positive cell when the discrimination information indicates that the cell 4 is not a positive cell. If necessary, the arithmetic unit 51 can store the information indicating the determination result in the storage unit 54 in association with the waveform data. The processing of S23 corresponds to a determination unit.

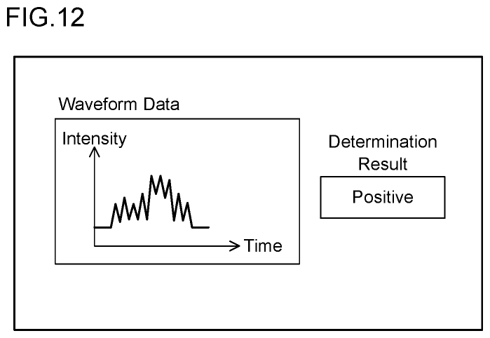

Then, the information processing device 5 displays the determination result on the display unit 56 (S24). FIG. 12 is a schematic diagram showing a display example of the determination result. On the display unit 56, for example, waveform data is displayed in the form of a graph, and the determination result as to whether or not the cell 4 is a positive cell is displayed in text. FIG. 12 shows an example in which the cell 4 is determined to be a positive cell. In S24, the arithmetic unit 51 can read the determination result from the storage unit 54, generate an image showing the waveform data and the determination result, and display the image on the display unit 56. The arithmetic unit 51 may generate an image from the waveform data and the determination result and display the image on the display unit 56 without storing information indicating the determination result in the storage unit 54. The arithmetic unit 51 may generate an image of the cell 4 based on the waveform data, and display the image of the cell 4 on the display unit 56 as well. The arithmetic unit 51 may also display on the display unit 56 information regarding the test sample 131 containing the cell 4 or information regarding the test subject 13 from whom the test sample 131 has been collected. Information regarding the determined cell 4 is displayed, and the user can check the information regarding the cell 4. S24 may be omitted.

Then, when it is determined that the cell 4 is not a positive cell (S25: NO), the information processing device 5 ends the process for determining cells. When it is determined that the cell 4 is a positive cell (S25: YES), the information processing device 5 sorts the cell 4 using the sorter 65 (S26). In S26, the arithmetic unit 51 transmits a control signal from the interface unit 57 to the sorter 65 to cause the sorter 65 to sort the cell 4. The sorter 65 sorts the cell 4 according to the control signal. For example, when the cell 4 has flowed to the sorter 65 through the channel 64, the sorter 65 applies an electric charge to the cell 4 and applies a voltage to change the movement path of the cell 4, thereby sorting the cell 4. After S26 ends, the information processing device 5 ends the process for determining cells.

The processing of S21 to S26 is performed for each of the cells contained in the plurality of test samples 131. The cells sorted by the processing of S21 to S26 are positive cells having specific morphological characteristics. The identification information of the tag attached to the sorted cells is analyzed, and the test subject 13 associated with the identification information is identified. At this time, detailed information on the sorted cells can be additionally obtained by further performing a biochemical test or a genetic test on the sorted cells. The identified test subject 13 is a test subject from whom the test sample 131 containing the positive cell has been collected. In this manner, a positive person is identified. When the positive cell is a cell having specific morphological characteristics due to the influence of a specific disease, the positive person is determined to be a patient with the specific disease. In this manner, it is possible to find a patient with a specific disease such as cancer among a plurality of test subjects 13.

As described in detail above, in the present embodiment, a classification model is trained by using training data including waveform data acquired for each cell contained in the first sample and the second sample. The first sample contains positive cells having specific morphological characteristics and other negative cells. The second sample does not contain positive cells and contains negative cells. The classification model outputs discrimination information indicating whether or not the cell is a positive cell when waveform data is input. Since positive cells are rare, it is not easy to obtain a large amount of waveform data of positive cells as training data. However, using the training data including information indicating whether the waveform data has been obtained from cells contained in the first sample or the second sample, a classification model can be generated by training using the MIL method. By using the classification model, discrimination information according to the waveform data can be obtained and it is furthermore possible to determine whether or not the cell is a positive cell based on the discrimination information. Therefore, it is possible to discriminate even rare positive cells.

When positive cells are cells that can be collected from a person with a specific disease such as cancer, it can be determined whether or not the person from whom the cells have been collected has the specific disease by determining whether or not the cells are positive cells. In the present embodiment, a tag having identification information for identifying a person from whom the cells have been collected is attached to the cells, and the person from whom the cells have been collected is identified based on the tag. When the cells are determined to be positive cells, the person from whom the positive cells have been collected can be identified based on the tag attached to the positive cells. Therefore, it is also possible to identify a person having a specific disease. In the present embodiment, an illustrative embodiment has been described in which a tag with identification information is attached to cells and a person from whom the cells have been collected is identified based on the tag. However, the method of identifying a person from whom cells have been collected is not limited to this. As another method, for example, it is possible to use a method in which observation of the cells contained in a test sample is performed in good order collectively for each person from whom the cells have been collected and the test sample is identified based on the observation order to identify a person from whom cells have been collected.

### Second Embodiment

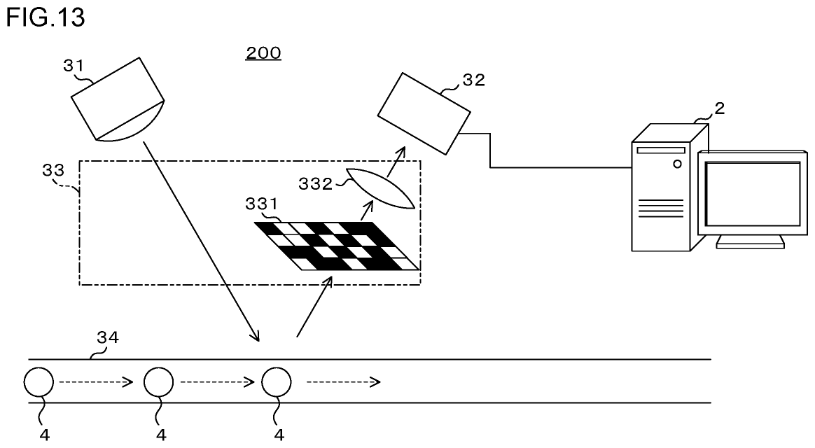

FIG. 13 is a block diagram showing a configuration example of a training apparatus 200 according to a second embodiment. In the second embodiment, the configuration of the optical system 33 is different from that in the first embodiment shown in FIG. 2. The configuration of components other than the optical system 33 is the same as that in the first embodiment. The light from the light source 31 is irradiated to the cell 4 without passing through the spatial light modulation device 331. The light from the cell 4 passes through the spatial light modulation device 331 and is condensed by the lens 332 to be incident on the detection unit 32. The detection unit 32 detects modulated light that is structured by passing the light modulated by the cell 4 through the spatial light modulation device 331. The configuration in which the modulated light from the cell 4 is structured by the spatial light modulation device 331 in the middle of the optical path from the cell 4 to the detection unit 32 as described above is also referred to as structured detection. The intensity of the modulated light from the cell 4 that is detected by the detection unit 32 changes over time due to the spatial light modulation device 331. The waveform data indicating the temporal change in the intensity of light from the cell 4, which is detected by the detection unit 32 by structured detection, includes compressed morphological information of the cell 4, as in the case of the structured illumination described above. That is, the waveform data indicating the temporal change in the intensity of light from the cell 4 that is detected by the detection unit 32 is observation data including the morphological information of the cell.

In the second embodiment as well, the training apparatus 200 can acquire waveform data indicating a temporal change in the intensity of light emitted from the cell 4. As in the first embodiment, the waveform data indicates the morphological characteristics of the cell 4. The optical system 33 includes optical components in addition to the spatial light modulation device 331 and the lens 332. In the structured detection, optical components used in the structured illumination in the first embodiment can be similarly used as the spatial light modulation device 331. In FIG. 13, descriptions of optical components other than the spatial light modulation device 331 and the lens 332 are omitted. In the second embodiment as well, the information processing device 2 generates the classification model 242 by performing the processing of S11 to S13 as in the first embodiment.

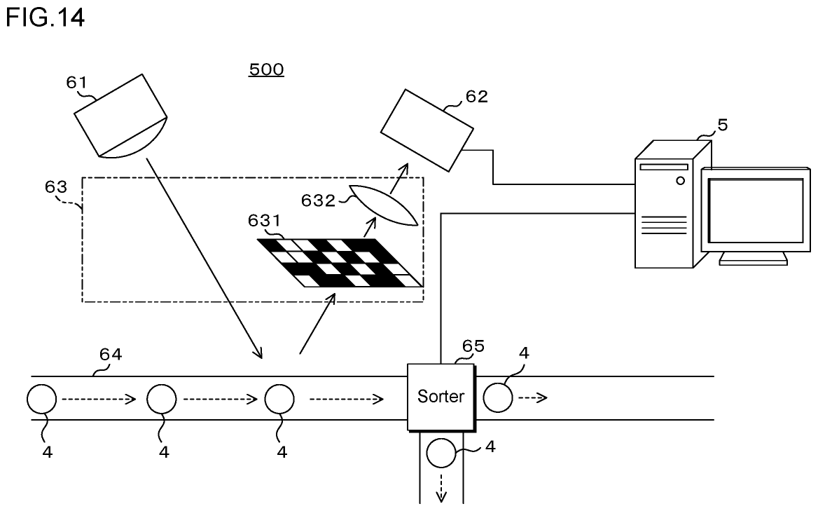

FIG. 14 is a block diagram showing a configuration example of a determination apparatus 500 according to the second embodiment. In the second embodiment, the configuration of the optical system 63 is different from that in the first embodiment shown in FIG. 9. The configuration of components other than the optical system 63 is the same as that in the first embodiment. The light from the light source 61 is irradiated to the cell 4 without passing through the spatial light modulation device 631. The light from the cell 4 passes through the spatial light modulation device 631 and is collected by the lens 632 to be incident on the detection unit 62. The detection unit 62 detects modulated light that is structured by passing the light modulated by the cell 4 through the spatial light modulation device 631. The waveform data indicating the temporal change in the intensity of light from the cell 4, which is detected by the detection unit 62 by structured detection, includes compressed morphological information of the cell 4. The intensity of light from the cell 4 changes according to the morphological characteristics of the cell 4 and also changes through the spatial light modulation device 631. That is, the waveform data indicating the temporal change in the intensity of light from the cell 4 that is detected by the detection unit 62 is observation data including the morphological information of the cell.

Also in the second embodiment adopting the configuration of the structured detection, the determination apparatus 500 can acquire waveform data indicating a temporal change in the intensity of light emitted from the cell 4. As in the first embodiment, the waveform data indicates the morphological characteristics of the cell 4. The optical system 63 includes optical components in addition to the spatial light modulation device 631 and the lens 632. In FIG. 14, descriptions of optical components other than the spatial light modulation device 631 and the lens 632 are omitted. In the second embodiment as well, the information processing device 5 performs cell discrimination and sorting by performing the processing of S21 to S26 as in the first embodiment. One of the training apparatus 200 and the determination apparatus 500 may have the same configuration as in the first embodiment.

In the second embodiment as well, a classification model can be generated by using training data that includes waveform data acquired for each cell contained in the first sample and the second sample and information indicating whether the waveform data has been obtained from cells contained in the first sample or the second sample. Using the classification model, it is possible to obtain discrimination information according to the waveform data and determine whether or not the cell is a positive cell having specific morphological characteristics. Therefore, it is possible to discriminate rare positive cells.

### Third Embodiment

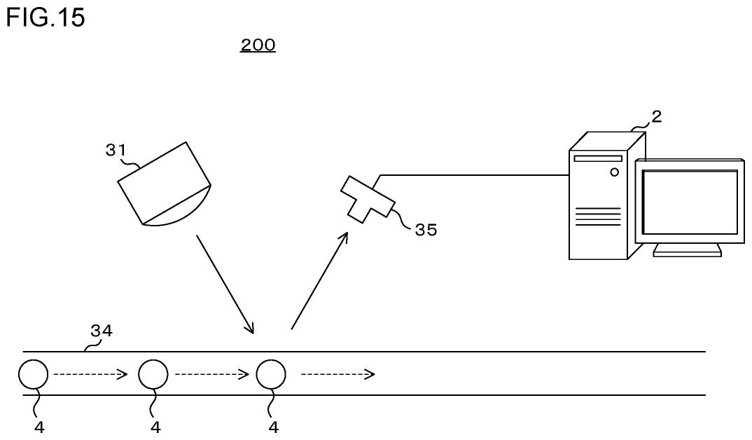

In the first and second embodiments, an example is shown in which the observation data indicating a result of observing a cell is waveform data including the morphological information of the cell. However, in a third embodiment, an example is shown in which the observation data including the morphological information of cells is a captured image obtained by imaging the cells. FIG. 15 is a block diagram showing a configuration example of a training apparatus 200 according to the third embodiment. In the third embodiment, the training apparatus 200 does not include the detection unit 32 and the optical system 33, but includes an imaging unit 35 for imaging the cell 4 moving through the channel 34. The light from the light source 31 illuminates the cell 4, the light from the cell 4 is incident on the imaging unit 35, and the imaging unit 35 creates a captured image by imaging the cell 4. For example, the imaging unit 35 is a camera having a semiconductor image sensor. The semiconductor image sensor is, for example, a CMOS (Complementary Metal-Oxide-Semiconductor) image sensor or a CCD (Charge Coupled Device) image sensor. The training apparatus 200 may include an optical system (not shown) for guiding light for imaging to the cell 4 to make the light incident on the imaging unit 35 efficiently. The imaging unit 35 is connected to the information processing device 2. The interface unit 27 is connected to the imaging unit 35. The interface unit 27 transmits and receives signals to and from the imaging unit 35. The other configuration of the training apparatus 200 is the same as that in the first embodiment.

In the third embodiment, the training apparatus 200 can acquire the captured image of the cell 4 by using the imaging unit 35. The captured image is observation data indicating the morphological characteristics of the cell 4. In the third embodiment, the classification model 242 is trained to output discrimination information indicating whether or not the cell 4 has specific morphological characteristics when data of a captured image is input.

In the third embodiment as well, the information processing device 2 performs the processing of S11 to S13 as in the first embodiment. In S11, the information processing device 2 acquires a captured image instead of waveform data. In S12, the information processing device 2 generates training data including a captured image and information indicating whether the captured image has been obtained from cells contained in the first sample 111 or the second sample 121. Each captured image is associated with information indicating whether the captured image has been obtained from cells contained in the first sample 111 or the second sample 121.

In S13, the information processing device 2 adjusts the calculation parameters of the classification model 242 so that discrimination information indicating that the cell is not a positive cell having specific morphological characteristics is output when the captured image obtained from the cell contained in the second sample 121 is input to the classification model 242. In addition, the arithmetic unit 21 adjusts the calculation parameters of the classification model 242 so that discrimination information indicating that the cell is a positive cell is output when some of the captured images obtained from a plurality of cells contained in one first sample 111 are input to the classification model 242 and discrimination information indicating that the cell is not a positive cell is output when the other captured images are input to the classification model 242. As described above, the classification model 242 is generated through the processing of S11 to S13.

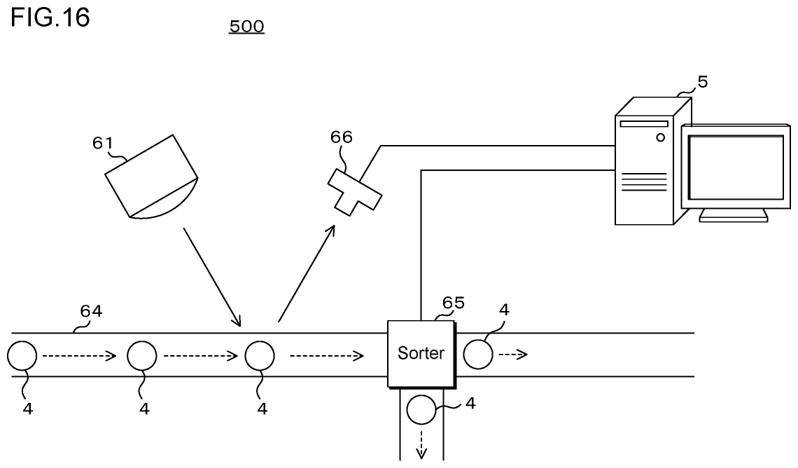

FIG. 16 is a block diagram showing a configuration example of a determination apparatus 500 according to the third embodiment. In the third embodiment, the determination apparatus 500 does not include the detection unit 62 and the optical system 63, but includes an imaging unit 66 for imaging the cell 4 moving through the channel 64. The light from the light source 61 illuminates the cell 4, the light from the cell 4 is incident on the imaging unit 66, and the imaging unit 66 creates a captured image by imaging the cell 4. The determination apparatus 500 may include an optical system (not shown) for guiding light for imaging to the cell 4 to make the light incident on the imaging unit 66 efficiently. The imaging unit 66 is connected to the information processing device 5. The interface unit 57 is connected to the imaging unit 66 and the sorter 65. The interface unit 27 transmits and receives signals to and from the imaging unit 66 and the sorter 65. The other configuration of the determination apparatus 500 is the same as that in the first embodiment.

In the third embodiment, the determination apparatus 500 can acquire the captured image of the cell 4 by using the imaging unit 66. In the third embodiment as well, the information processing device 5 performs the processing of S21 to S26 as in the first embodiment. In S21, the information processing device 5 acquires a captured image instead of waveform data. In S22, the information processing device 5 inputs the captured image instead of waveform data to the classification model as observation data indicating the morphological characteristics of the cell 4. In S24, the information processing device 5 displays the captured image and the determination result on the display unit 56. The information processing device 5 performs cell identification and sorting by performing the processing of S21 to S26.

In the third embodiment, a classification model can be generated by using training data that includes a captured image acquired for each cell contained in the first sample and the second sample and information indicating whether the captured image has been obtained from the cell contained in the first sample or the second sample. Using the classification model, it is possible to obtain discrimination information according to the captured image and determine whether or not the cell is a positive cell having specific morphological characteristics. Therefore, it is possible to discriminate rare positive cells.

In the first to third embodiments described above, the first sample 111, the second sample 121, and the test sample 131 are specimens collected from a person, but the first sample 111, the second sample 121, and the test sample 131 may be specimens collected from those other than a person. Alternatively, the first sample 111, the second sample 121, and the test sample 131 may be samples created by using a method other than collecting a specimen. In the first to third embodiments, an illustrative embodiment is shown in which the determination apparatus 500 includes the sorter 65 to sort cells. However, the determination apparatus 500 may not include the sorter 65. In this illustrative embodiment, the information processing device 5 omits the processing of S25 and S26. In the first to third embodiments, an illustrative embodiment is shown in which the training apparatus 200 and the determination apparatus 500 are different. However, the training apparatus 200 and the determination apparatus 500 may be partially or entirely the same. For example, the information processing device 2 may also be used as the information processing device 5.

In the first to third embodiments, an example in which particles are cells has been described. However, in the classification model generation method and the particle determination method, particles other than cells may be handled. Particles are not limited to biological particles. For example, particles targeted in the classification model generation method and the particle classification method may be microorganisms such as bacteria, yeast, and plankton, tissues within organisms, organs within organisms, or fine particles such as beads, pollen, and particulate matter.

The present invention is not limited to the content of the above-described embodiments, and various changes can be made within the scope of the claims. That is, embodiments obtained by combining technical means appropriately changed within the scope of the claims are also included in the technical scope of the present invention.

### (Note 1)

A non-transitory recording medium recording a computer program causing a computer to execute processing of:
acquiring observation data indicating a result of observing a particle;
inputting the acquired observation data to a classification model, which outputs discrimination information indicating whether or not a particle has specific morphological characteristics when the observation data indicating a result of observing the particle is input,
acquiring the discrimination information output from the classification model; and
determining whether or not the particle related to the observation data has the specific morphological characteristics based on the acquired discrimination information,
wherein the classification model is trained by using training data, which includes observation data indicating a result of observing a particle and information indicating whether the observation data has been obtained from a particle contained in a first sample or a second sample, for each particle contained in the first sample containing a mixture of particles having the specific morphological characteristics and other particles and the second sample that does not contain particles having the specific morphological characteristics but contains only the other particles.

### (Note 2)

An information processing device, comprising:
a processor; and
a memory, wherein the processor is operable to:
acquire, for each particle contained in a first sample containing a mixture of particles having specific morphological characteristics and other particles and a second sample that does not contain particles having the specific morphological characteristics but contains only the other particles, observation data indicating a result of observing the particle; and
generate a classification model, which outputs discrimination information indicating whether or not a particle has the specific morphological characteristics when the observation data indicating a result of observing the particle is input, by training using training data including the observation data and information indicating whether the observation data has been obtained from a particle contained in the first sample or the second sample.

### (Note 3)

An information processing device, comprising:
a processor; and
a memory, wherein the processor is operable to:
acquire observation data indicating a result of observing a particle;
input the acquired observation data to a classification model, which outputs discrimination information indicating whether or not a particle has specific morphological characteristics when the observation data indicating a result of observing the particle is input,
acquire the discrimination information output from the classification model; and
determine whether or not the particle related to the observation data has the specific morphological characteristics based on the acquired discrimination information,
wherein the classification model is trained by using training data, which includes observation data indicating a result of observing a particle and information indicating whether the observation data has been obtained from a particle contained in a first sample or a second sample, for each particle contained in the first sample containing a mixture of particles having the specific morphological characteristics and other particles and the second sample that does not contain particles having the specific morphological characteristics but contains only the other particles.

It is to be noted that, as used herein and in the appended claims, the singular forms “a”, “an”, and “the” include plural referents unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

It is to be noted that the disclosed embodiment is illustrative and not restrictive in all aspects. The scope of the present invention is defined by the appended claims rather than by the description preceding them, and all changes that fall within metes and bounds of the claims, or equivalence of such metes and bounds thereof are therefore intended to be embraced by the claims.

1-10. (canceled)

11. A classification model generation method, comprising:
acquiring, for each particle contained in a first sample containing a mixture of particles having specific morphological characteristics and other particles and a second sample that does not contain particles having the specific morphological characteristics but contains only the other particles, observation data indicating a result of observing the particle; and
generating a classification model, which outputs discrimination information indicating whether or not a particle has the specific morphological characteristics when the observation data indicating a result of observing the particle is input, by training using training data including the observation data and information indicating whether the observation data has been obtained from a particle contained in the first sample or the second sample.

12. The classification model generation method according to claim 11,
wherein the observation data is waveform data indicating a temporal change in an intensity of light emitted from a particle irradiated with light by a structured illumination or waveform data indicating a temporal change in an intensity of light detected by structuring light from a particle irradiated with light.

13. The classification model generation method according to claim 11,
wherein the first sample is a specimen collected from a person who has a specific disease, and
the second sample is a specimen collected from a person who does not have the specific disease.

14. A particle determination method, comprising:
acquiring observation data indicating a result of observing a particle;
inputting the acquired observation data to a classification model, which outputs discrimination information indicating whether or not a particle has specific morphological characteristics when observation data indicating a result of observing the particle is input,
acquiring the discrimination information output from the classification model; and
determining whether or not the particle related to the observation data has the specific morphological characteristics based on the acquired discrimination information,
wherein the classification model is trained by using training data, which includes observation data indicating a result of observing a particle and information indicating whether the observation data has been obtained from a particle contained in a first sample or a second sample, for each particle contained in the first sample containing a mixture of particles having the specific morphological characteristics and other particles and the second sample that does not contain particles having the specific morphological characteristics but contains only the other particles.

15. The particle determination method according to claim 14, further comprising:
outputting information regarding the particle for which the determination has been made.

16. The particle determination method according to claim 14,
wherein a particle from which the observation data is to be acquired is collected from a person,
a tag having identification information for identifying a person from whom a particle has been collected is attached to the particle, and
when the particle related to the observation data has the specific morphological characteristics, a person from whom the particle has been collected is identified based on the identification information of the tag attached to the particle related to the observation data.

17. A non-transitory recording medium recording a computer program causing a computer to execute processing of:
acquiring, for each particle contained in a first sample containing a mixture of particles having specific morphological characteristics and other particles and a second sample that does not contain particles having the specific morphological characteristics but contains only the other particles, observation data indicating a result of observing the particle; and
generating a classification model, which outputs discrimination information indicating whether or not a particle has the specific morphological characteristics when the observation data indicating a result of observing the particle is input, by training using training data including the observation data and information indicating whether the observation data has been obtained from a particle contained in the first sample or the second sample.
